(19) United States
(12) Patent Application Publication
(10) Pub. No.: US 2011/0009892 A1
(43) Pub. Date: Jan. 13, 2011

(54) COVERS FOR TISSUE ENGAGING MEMBERS

(75) Inventor: Stephen J. SCHOENBERG, Auckland (NZ)

Correspondence Address: YOUNG BASILE, 3001 WEST BIG BEAVER ROAD, SUITE 624, TROY, MI, 48084, US

(73) Assignee: ABBOTT DIABETES CARE INC., Alameda, CA (US)

(21) Appl. No.: 12/885,789

(22) Filed: Sep. 20, 2010

Related U.S. Application Data
(62) Division of application No. 11/396,355, filed on Mar. 31, 2006.

(51) Int. Cl. A61B 17/34; A61B 17/32

(52) U.S. Cl. 606/181; 606/185; 606/167

(57) ABSTRACT

Moveable tissue engaging members and methods of fabricating and using the same are provided.

### CROSS REFERENCE TO RELATED APPLICATIONS

This application is a divisional application of and claims priority from U.S. patent application Ser. No. 11/396,355 filed Mar. 31, 2006, which is hereby incorporated by reference in its entirety.

### BACKGROUND OF THE INVENTION

Disease prevention and/or health maintenance sometimes requires the injection of various agents, such as medicaments, vaccines, and the like, as well as the routine analysis of blood. Injection devices, such as syringes, are well known and often used by the medical and dental communities for the delivery of various agents to a subject. Additionally, the analysis of bodily fluid samples (e.g., blood) is a common practice used in the diagnosis of disease conditions and the daily monitoring and maintenance of health. A lancing device is an instrument often used to draw bodily fluid when only a small amount of blood is required for analysis, for instance, where only a few microliters or less of fluid need be drawn. The utilization of a lancing device is particularly useful when the analysis of a fluid sample is to be performed soon after collection, for instance, when utilized in the daily monitoring of blood glucose levels.

Both syringes and lancing devices include a tissue engaging member that is configured for piercing the skin. However, the sharp and/or piercing nature of the tissue engaging member of a syringe or lancing device poses a threat of injury and a risk of the spread of disease due to accidental skin pricks. Therefore, in order to avoid accidental injury and prevent the spread of disease, there are several protective measures that are necessary to ensure the safe use and disposal of syringes and lancing devices. First, the skin engaging portion of the tissue engaging member (e.g., the needle or lance) needs to be sterilized and maintained in a contamination-free condition prior to use. Second, the needle or lance needs to be protected from accidental pricks, especially after use. An additional protective measure would be to prevent the reuse of the device after its initial utilization.

There is a continued need for protective covers that protect a tissue engaging member, for instance, of a syringe or sample collecting device, from contamination prior to use and reduce the risk of injury due to accidental contact with an exposed tissue engaging member after use, as well as prevent the tissue engaging member from being reused.

### SUMMARY OF THE INVENTION

Various embodiments of the present invention include a retractable protective cover or cap for protecting a tissue engaging member of a device. Generally, embodiments of the invention relate to a retractable cover that may be movably associated with a base member, wherein the base member holds a tissue engaging member, and the cover is adapted to cover and protect at least a distal portion of the tissue engaging member from contamination, accidental contact and/or reuse. In certain embodiments the cover is configured for being locked in place about the tissue engaging member to prevent unintended contact with the user. Specifically, in certain embodiments, the invention relates to a retractable and/or lockable cover for a sample collecting device (e.g., a lancet) or an injection device (e.g., a syringe), devices that include the retractable and lockable cover, as well as to methods of manufacturing and using such devices.

In one embodiment, a cover configured for protecting a distal portion of a tissue engaging member, for instance a piercing or cutting member, is provided. The cover includes a soft material body, for instance, a polymer body that is adapted for receiving the tissue engaging member. The soft material body may be at least partially bounded by a structural element that is configured for moveably attaching to a base or holding member which holds the tissue engaging member. In certain embodiments the cover is configured for interacting with the base member in a manner that allows the cover to be locked in place while covering the tissue engaging member so as to prevent accidental contact and/or reuse.

In another embodiment, a tissue engaging device is provided (for instance, a medicament delivery device or a sample collecting device). The tissue engaging device includes a tissue engaging member (e.g., a piercing or cutting member), the aforementioned retractable and/or lockable cover, and a base member, for instance, a base member that is adapted for both receiving the moveable cover and for holding the tissue engaging member. In certain embodiments, the tissue engaging device is a sample collecting device, such as a lancing device, which is configured to collect a blood sample from a subject. In certain embodiments the tissue engaging device is a delivery or injection device, e.g., a syringe, configured to deliver or inject a flowable solution (e.g., a medicament) to a subject. In additional embodiments, methods for fabricating and using the retractable protective covers and devices of the invention are also provided.

### BRIEF DESCRIPTION OF THE DRAWINGS

FIG. 1A is a perspective view of one embodiment of a retractable, protective cover of the invention and a lancing-type device, wherein the cover is shown detached from the lancing device.

FIG. 1B is a perspective view of the cover and lancing device of FIG. 1A shown operatively coupled with the cover in a distally extended and unlocked position.

FIG. 1C is a perspective view of the device and cover of FIG. 1B shown operatively coupled with the cover in a retracted position, wherein a lance is exposed from a distal end of the cover.

FIG. 1D is a perspective view of the device and cover shown operatively coupled with the cover in an extended and locked position.

FIGS. 2A-2C illustrate another embodiment of a lancing device and cover shown in operative connection. The cover is in an extended (2A), retracted (2B), and extended and locked position (2C).

FIGS. 3A-3C illustrate another embodiment of a lancing device and cover shown in operative connection. The cover is in an extended (3A), retracted (3B), and extended and locked position (3C).

### DETAILED DESCRIPTION OF THE INVENTION

Embodiments of the present invention are directed to a retractable and protective cover for protecting and covering at least a distal portion of a tissue engaging member of a medical or dental device that contains a cutting, piercing and/or other tissue contacting member on a distal portion of the device. Generally, although the cover is suitable for protecting any tissue engaging member on a distal portion of a medical or dental device, it is particularly suitable for use with sample collecting or delivery devices that contain a sharp or pointed tissue engaging member such as a blade, a lance, or a needle on a distal portion of the device, for instance, a lancing device, a syringe, a scalpel, or the like. Accordingly, in certain embodiments, a retractable cover of the invention is configured for use with a sample collecting device, for instance, a blood collecting device, such as a lancet, or a fluid delivery device, for instance, a syringe, wherein the cover is moveably associated with a body, barrel or handle of the device and is adapted for contacting and protecting a tissue engaging member being held by the body of the device. In certain embodiments, the cover protects the tissue engaging member by being locked in place while covering the tissue engaging member thereby preventing accidental contact with the tissue engaging member.

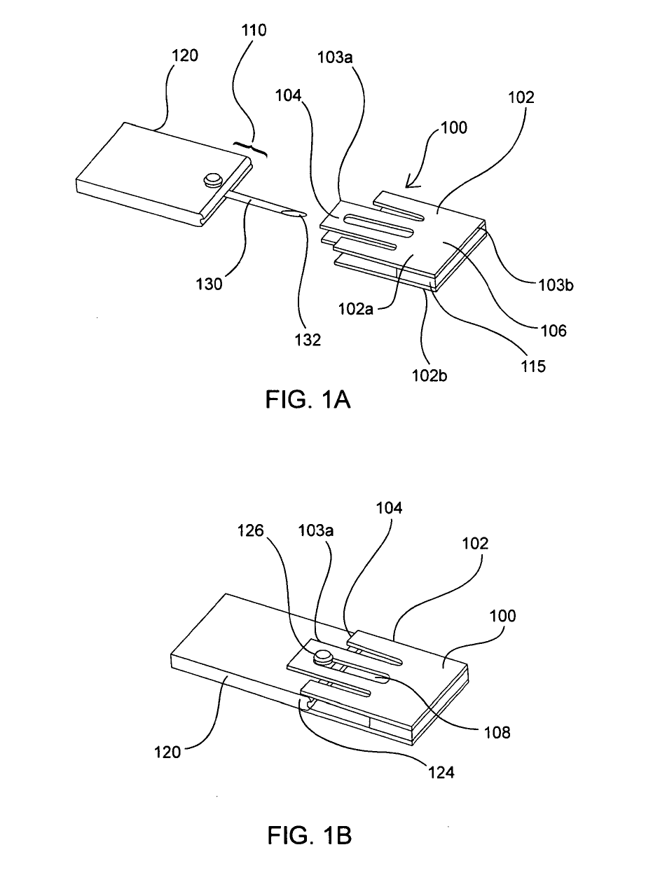

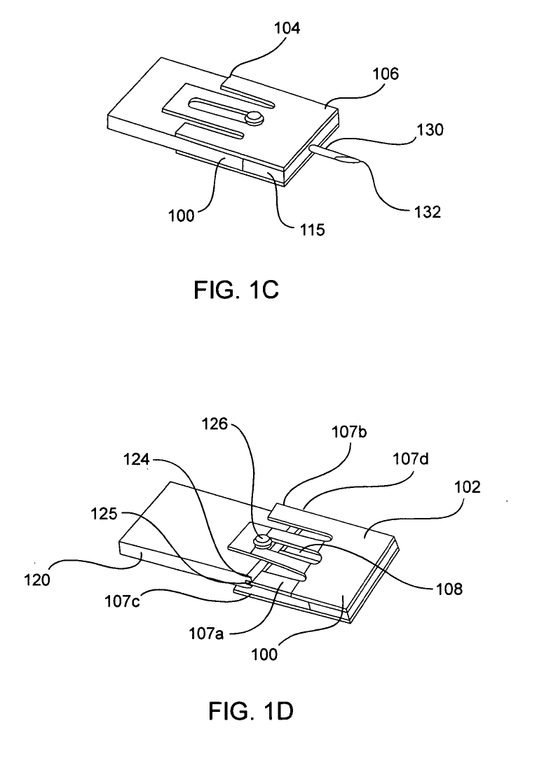

Various embodiments of the invention are exemplified with reference to FIGS. 1 to 6. It is to be understood, however, that although certain configurations are set forth in relation to the following figures, this is not meant to limit the scope of the invention in any way. Therefore, although, the covers and other various components of the invention are set forth herein below with reference to use in conjunction with a tissue engaging device, for instance, a lancing device, it is to be understood that the protective covers of the invention can easily be adapted for use with other medical and dental devices that contain a cutting, piercing and/or intimate tissue contacting member on a distal portion of the device (such as, a syringe, other hypodermic needle containing device, scalpel, or the like) with the appropriate modifications.

With reference to FIGS. 1A-1D, a retractable and protective cover of the invention is hereby provided. A cover (100) of the invention is configured for being movably associated with a holding member (120), for instance, a base member that holds a tissue engaging member (130). The cover (100) is generally configured for fitting over and covering at least a distal portion (132) of the tissue engaging member (130) and is adapted for moveably associating with and/or connecting to the holding member (120).

The cover (100) may include one or more elements. For example, in certain embodiments, a cover may include a first element that is a soft material body (115), and/or a second element that is a structural element (102) that is provided to give form and shape to the soft material body (115). The soft material body (115) is configured for contacting, receiving and encasing at least a distal portion (132) of a tissue engaging member (130), and thereby protects the tissue engaging member (130) from contamination and unintentional use.

The soft material body may be made of any soft material, for instance, a soft polymer material that is capable of covering at least a distal portion of a tissue engaging member and protecting it from contamination. A soft polymer body of the invention should be fabricated from a polymer that is capable of undergoing a sterilization process, e.g., while covering the tissue engaging member, and maintaining functional integrity after the sterilization process. In certain embodiments, the soft polymer body is an elastomer. A variety of elastomers may be used to form the soft polymer body, for instance, a suitable elastomer may be fabricated from one or more of a rubber, a vulcanized rubber, polyisoprene, polyisobutylene, butyl rubber, polybutadiene, polystyrene, Styrene Butadiene Rubber, acrylonitrile, nitrile rubber, chloroprene rubber, polychloroprene, neoprene, silicone, vinylidene fluoride, hexafluoropropylene, fluorosilicone rubber, ethylene propylene rubber, polyethylene, polyurethane rubber, resilin, polyacrylic rubber, epichlorohydrin rubber, polysulfide rubber, chlorosulfonated polyethylene, or the like.

In certain embodiments, the soft polymer body of the invention is a thermoplastic elastomer, such as, but not limited to one or more of: a styrene oligoblock copolymer (so-called TPE-S), a thermoplastic polyolefin (TPE-O), a thermoplastic polyurethane (TPE-U), a thermoplastic copolyester (TPE-E), a thermoplastic copolyamide (TPE-A), elastomers based on styrene-ethylene-butylene-styrene-polymers, an acrylonitrile butadiene styrene, an acrylic, celluloid, cellulose acetate, ethylene-vinyl acetate, ethylene vinyl alcohol, fluoroplastics, PTFEs (including: FEP, PFA, CTFE, ECTFE, ETFE), an ionomer, a liquid crystal polymer, an acetal polyacrylate, a polyacrylonitrile, an acrylonitrile, a polyamide, nylon, a polyamide-imide, a polyaryletherketone, a ketone, a polybutadiene, a polybutylene, a polycyclohexylene dimethylene terephthalate, a polyketone, a polyester, a polyethylene/polythene/polyethene, a polyetheretherketone, a polyetherimide, a polyethersulfone, a polysulfone, a polyethylenechlorinates, a polyimide, a polylactic acid, a polymethylpentene, a polyphthalamide, a polypropylene, a polysulfone, a polyvinyl chloride, a spectralon, or the like.

As noted above, a cover (100) may include a structural element (102) to provide form and shape to the cover. A structural element may include at least two sides (102a and 102b) which at least partially bound the soft material body (115). The structural element (102) may include more or less than two sides or portions thereof, for instance, one, three, four, five, or more sides so as to further bound and shape the soft material body, for instance, the structural element may be round. The two or more sides of the structural element may be joined or separated, but are typically on opposing sides of the soft material body. The structural element may be substantially rigid so as to facilitate the use, handling, gripping, packaging, storing, etc., of the device. As shown with reference to FIG. 1A, the structural element (102) includes two sides 102a and 102b, which bound the soft material body (115) on front and back sides. The structural element has both a proximal and a distal portion (103a and 103b, respectively). The soft material body (115) may be positioned at, and bound by, the distal portion (103b) of the structural element (102).

It is to be noted, that reference to the terms “proximal” or “distal” is with respect to how the device is held and used. For instance, the term “proximal” references a portion of the device that is closer to the end of the device that is to be held by a user when using the device. In other words, the “proximal end” of the device is the end of the device that is furthest away from the tissue engaging portion. Accordingly, the term “distal” references a portion of the device that is further away from the end of the device that is to be held by a user when using the device. In other words, the “distal end” of the device is the end of the device that is closest to the tissue engaging portion of the device.

As shown with reference to FIG. 1A, in this embodiment both the proximal and distal portions (104 and 106, respectively) of the cover (100) contain ends that are unbounded by the structural element (102). That is, the proximal (103a) and distal (103b) portions of the structural element (102) are relatively open so as to allow the soft material body (115) to receive and pass at least a distal portion (132) of the tissue engaging member (130). It is to be noted that embodiments may be configured otherwise, e.g., the distal portion may be relatively closed, etc. Accordingly, when covered the tissue engaging member (130) is inserted through the proximal portion (103a) of structural element (102) and is embedded within the soft material body (130). For use, the cover (100) is retracted and the tissue engaging member (130) pierces through the distal portion of the soft material body (115) and is thereby exposed. As described in greater detail herein below, the proximal portion (103a) of the structural element (102) is further configured for moveably communicating with and/or attaching to the base or holding member (120).

Although as shown in FIG. 1A, the proximal and distal portions (103a and 103b, respectively) contain unbounded ends, one or more of the ends and/or sides can be bounded so long as they are configured for receiving and passing a tissue engaging member (130) there through. That is, the end of the proximal portion is adapted for allowing the tissue engaging member (130) to pass through the proximal end of the structural element (102) and into the soft material body (115) that is bounded by the structural element (e.g., at the distal portion of the structural element (103a)). Further, the end of the distal portion (103b) of the structural element (102) is also adapted for allowing the tissue engaging member (130) to pass through the soft material body (115) and out of the distal end of the distal portion (103b) of the structural element (102). Hence, if one or more of the distal or proximal ends of the structural element (102) are to be bounded, then the structural element (102) may further include a passage (for instance, a hole or the like) through which the tissue engaging member (130) passes into and/or out of the structural element (102). Accordingly, for use, the tissue engaging member (130) pierces through the distal portion of the soft material body (115) and passes through the passage (e.g., a hole) in the structural element (102).

The structural element may be of any desirable shape so as to produce a close, intimate contact between the soft material body and the tissue engaging element, for instance, square, rectangular, round, circular, conical, or the like. In many embodiments, the structural element is substantially rigid although in certain embodiments it may be semi-rigid or flexible. For instance, in certain embodiments, both the structural element and the soft material body are fabricated from soft materials and are therefore soft and/or flexible. Accordingly, the structural element may be made of any material capable of bounding the soft material body and giving the soft material body a desired shape. The structural element should be made of a material that is capable of undergoing a sterilization process and not losing its shape or breaking down. For instance, in certain embodiments, the soft material body may be a laminant and the structural element may be made of material that provides the requisite rigidity to the device, e.g., surgical grade metal or plastic. Suitable metals from which the structural element may be fabricated include medical grade steel, stainless steel, aluminum alloys, and the like. Suitable plastics from which the structural element may be fabricated include polycarbonate, polyvinyl chloride, polyethylene terephthalate, polybutylene terephthalate, polypropylene, polyethylene, polymethylmethacrylate, polystyrene, ABS, and the like. In certain embodiments, the structural element is a polymer that is coextruded with the soft material body.

The cover (100) includes both a proximal portion (104) and a distal portion (106). The proximal portion (104) of the cover (100) is configured for both receiving at least a distal portion (132) of a tissue engaging member (130) and moveably associating and/or attaching to a base or holding member (120), which holds the tissue engaging member (130). The cover (100) and the holding member (120) may be associated with and/or coupled to one another in any manner so long as the cover (100) can move axially and/or rotationally along the tissue engaging member (130). The engagement and movement between the retractable and protective cover (100) and the base or holding member (120) is illustrated with reference to FIGS. 1B-1D.

As can be seen with reference to the embodiments of FIG. 1B, the cover (100) is moveably attached (e.g., slideably linked) to the base member (120). As stated above, the nature of this attachment may take any form so long as the cover (100) can move axially and/or rotationally along the tissue engaging member (130). That is, the communication between the cover (100) and the base member (120) should be such that the cover (100) is capable of moving axially (e.g., longitudinally) along the tissue engaging member (130) from an extended position (as shown in FIG. 1B) to a retracted position (as shown in FIG. 1C).

In one embodiment, to facilitate the communication between the cover (100) and the base member (120) and to allow the cover (100) to move in the requisite directions, the cover (100) is sized and configured to allow at least the proximal portion (104) of the cover (100) to fit and move over at least a distal portion (124) of the base member (120). Accordingly, the configuration of the cover (100) and the base member (120) should be such that the cover (100) is allowed to slide along the tissue engaging member (130) and at least a proximal portion of the cover (104) is allowed to slide over at least a distal portion of the base member (124). Hence, the engagement between the cover (100) and the base member (120) should provide a snug fit, but not so tight a fit as to prevent the cover (100) from sliding downward toward and/or over at least a proximal portion (124) of the base member (120).

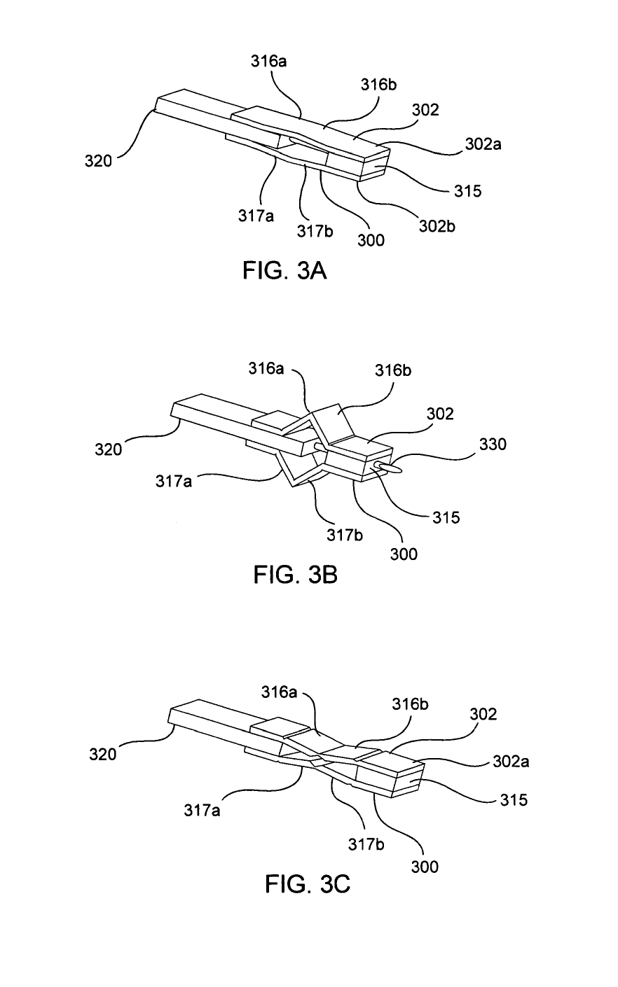

Although in the above embodiment the cover is configured for allowing at least a proximal portion of the cover to slide over at least a distal portion of the base member, it is understood that the nature of the attachment between the cover and base member may be such that although the cover is allowed to slide toward and/or away from the base member, a portion of the cover may not actually slide over the base member. For instance, in one embodiment, at least a portion of the cover is a flexible or linking member that is attached to the base member in such a way that when the cover is moved from an extended configuration to a retracted configuration the flexible or linking member of the cover is expanded (e.g., outward) rather than sliding over the base element. See, for instance, the embodiment of FIG. 3.

The base or holding member (120) is configured for holding the tissue engaging member (130). The base member may be fabricated out of any suitable material and may be configured to include one or more removable cartridges, sensors, motors, meters, test strips and the like. Accordingly, the base member may include a lumen into which one or more of the removable cartridges, sensors, motors, meters, test strips and the like are fitted. A motor for use with a device of the invention may be configured for moving a tissue engaging member from a retracted and covered position to an extended position whereby a distal portion of the tissue engaging member pierces through the cover.

The engagement between the cover (100) and the base member (120) may further be such that the cover (100) may be removably attached to the base member (120), but in a manner that prevents accidental detachment of the cover (100) from the base member (120). For instance, with reference to FIG. 1B, the proximal portion (203A) of the structural element (102) of the cover (100) may include one or more slots or grooves (108) by which one or more engagement members (126), such as a pin or button, of the base member (120) couples the cover (100) to the base member (120). The portion of the structural element forming the slot(s)(108) may be fabricated from any suitable material, e.g., a flexible material that allows the sides of the slot (108) to be flexed so as to allow the cover (100) to be snapped into place about the base member (120) via the engagement member (126). Additionally, the edges of the slot may be compressible and the slot may have a tapered configuration so as to provide a fixed engagement between the engagement member and the slot. The engagement member (126) may be such that it is immovable and thereby prevents the cover (100) from being detached once snapped in place, or the engagement member (126) may be depressable and thereby allow the cover (100) to be detached from the base member (120).

In certain embodiments, the engagement member (126) and the slot (108) are configured so that the cover (100) is capable of sliding downward toward the base member (120) from an extended to a retracted position. That is, the slot (108) allows the cover (100) to slide downward relative to the engagement member (126) along the length of the slot (208). It is to be understood that although the moveable attachment of the cover to the base member has been described with reference to a slot or groove and engagement member, other configurations for moveably engaging the cover to the base member are well within the scope of the present invention. For instance, the engagement member may simply be a tether that removably or non-removably engages the cover with the base member. Accordingly, the engagement of the cover to the base member may be of any configuration so long as the cover is allowed to move proximally and distally with respect to the base member, along the longitudinal axis of a tissue engaging member, and is prevented from accidental detachment from the base member.

As can be seen with reference to FIGS. 1B and 1C, the cover (100) is configured for moving from an extended position (as seen in FIG. 1B) to a retracted position (as seen in FIG. 1C). As seen in FIG. 1B when the cover (100) is in an extended position, at least a distal portion of the tissue engaging member (not shown) is covered and thereby protected by the cover (100). That is, in an extended position, at least a distal portion of tissue engaging member is encased, covered, or embedded within the soft material body (115) of the cover (100). The soft material body (115) is such that when the tissue engaging member is embedded within the soft material body (115) the tissue engaging member is protected from contamination and accidental contact, additionally, the tight seal of the soft material body enables the lancet to remain sterile. As will be described in greater detail herein below, the cover (100) and/or base member (120) may further include a locking mechanism to lock the cover in an extended position before or after use. As seen in FIG. 1C, when the cover (100) is in a retracted position, at least a distal portion (132) of the tissue engaging member (130) is exposed. For instance, when the cover (100) is in a retracted position, a distal portion of the tissue engaging member is exposed and extends beyond the end of the distal portion (106) of the cover (100).

As can be seen with reference to FIG. 1D, the association between the cover (100) and the base member (120) is such that the cover (100) may be locked in place. Any locking means may be used to lock the cover (100) so as to prevent the cover from moving axially along the tissue engaging member (not shown) from an extended to a retracted position. When the cover is in an extended and locked position, the cover completely encloses at least a distal portion of the tissue engaging member, preventing it from being exposed, and thereby protecting the tissue engaging member from accidental contact, which consequently prevents accidental pricks, reuse and the spread of disease.

For instance, the engagement member (126) may be configured for moving from an unlocked to a locked position, wherein in the unlocked position the cover (100) may retract by sliding toward the base member (120). In this position, the unlocked engagement member (126) associates with the cover (100) within the slot (108) so as to allow the cover (100) to move relative to the base (120). In a locked position, the engagement member (126) associates with the cover (100) within the slot (108) so as to prevent the cover from moving relative to the base (120). For instance, in one embodiment the engagement member may be twisted or torqued from an unlocked to a locked position or depressed so as to move from a locked to an unlocked position (or vice versa).

In another embodiment, the structural element (102) of the cover (100) includes at least one flange or leg member. The flange member may be an internal or an external component of the structural element (102). For instance, in one embodiment, the one or more flange members is an internal element that is collapsible toward the tissue engaging member when subjected to an applied force. In this embodiment, the tissue engaging member includes a transverse groove into which the flange member(s) collapse when an appropriate force is applied. Once the flange member(s) are collapsed about the groove of the tissue engaging member the cover is locked into place and thereby prevented from further axial movement along the tissue engaging member. Accordingly, when in a collapsed and locked position the cover is locked in place around at least a distal portion of the tissue engaging member preventing it from being exposed.

The structural element (102) may include one or more external flange members (107). As seen in FIG. 1D the structural element (102) includes four external flange members (107a-d). The base member (120) further includes at least one groove (125) along a distal end of a distal portion (124) of the base member (120). For instance, the groove (125) may be a transverse groove that runs perpendicular to the longitudinal axis of the tissue engaging member along the distal end of the base member (120). In this embodiment, the cover (100) may be locked in place about at least a distal portion of the tissue engaging member by being twisted or torqued rotationally about the longitudinal axis of the tissue engaging member. In this manner, one or more of the external flange or leg members (107a-d) may be displaced into the transverse groove (125) thereby locking the cover (100) in place about at least a distal portion of the tissue engaging member and preventing the cover (100) from moving from the extended to a contracted position. In this or any other embodiment, the cover may further be prevented from moving rotationally about the longitudinal axis of the tissue engaging member once the cover is in a locked configuration and may also be prevented from being detached from the base member.

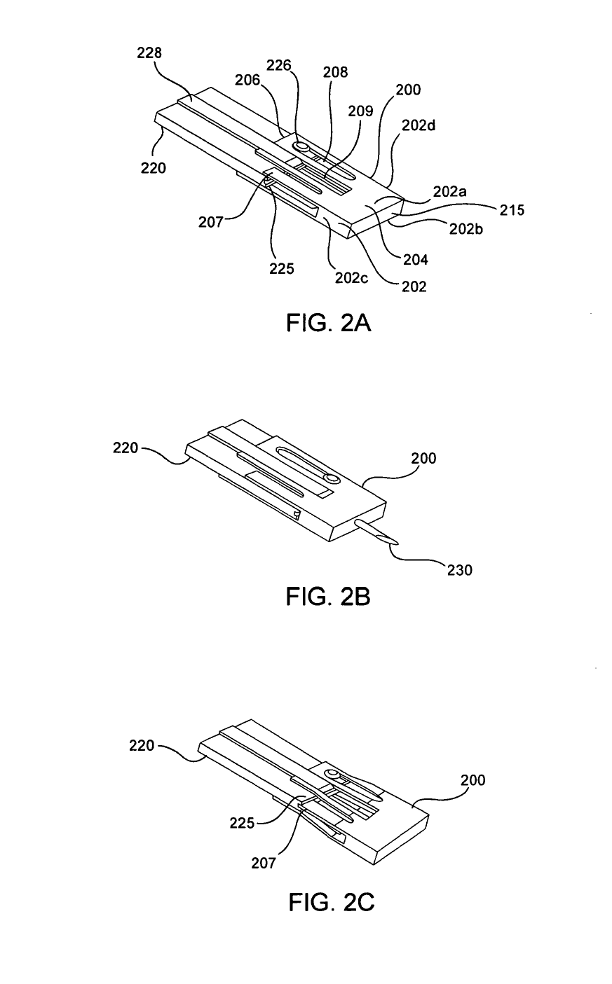

FIGS. 2A-2C illustrate another embodiment of a tissue engaging device of the invention, for instance, a lancing device. The tissue engaging device includes a base member (220), a tissue engaging member (230), and a cover (200). The cover (200) includes a structural element (202) that contains four sides (202a, 202b, 202c, and 202d) which bound a soft material body (215) on a front, back, left, and right side. The structural element (202) of the cover (200) does not bound the soft material body (215) on the proximal and distal ends of the cover (204 and 206, respectively).

The cover (200) further includes a bounded slot (208) and an opened slot (209). The base member (220) contains an engagement element (226) that fits into the enclosed slot (208) and a raised portion (228) that fits into the opened slot (209). The structural element (202) further includes at least one external flange or leg members (207). The cover (200) is slideably linked to the base member and is configured for sliding along the longitudinal axis of the tissue engaging member (230) from an extended position, as seen in FIG. 2A, wherein the tissue engaging member (230) is covered and protected by the cover, to a retracted position, as seen in FIG. 2B, wherein the tissue engaging member (230) is exposed and extends beyond the cover (200). As seen in FIG. 2C, the base member (220) further includes a transverse groove (225) into which one or more of the external flange member(s) (e.g., 207) may be displaced so as to lock the cover (200) in an extended position, thereby covering the tissue engaging member (230) and preventing any further axial movement of the cover (200).

Although, the above embodiment has been described with respect to the cover containing an enclosed slot within which a portion of the base member slides, it is understood that the base member may include one or more slots or channels into which the structural element of the cover is fitted thereby allowing the cover to slide relative to the base member within the one or more channels of the base member. Accordingly, where one or more channels or slots are included in the base member, the one or more slots may be deformable or otherwise be collapsible or contain a collapsible element so as to prevent the movement of the cover through the slot or channel once the collapsible portion is collapsed, for instance, in response to an applied force.

FIGS. 3A-3C illustrate an embodiment of a tissue engaging device of the invention. In this embodiment the device includes a base member (320), a cover (300), and a tissue engaging member (330). The cover (300) is movably associated with the base member (320). The cover (300) includes a structural element (302) that contains at least two sides (302a and b) which bound the soft material body (315) on a front (302a) and a back (302b) side. The structural member also includes a compressible portion. The compressible portion may have a variety of configurations, so long as it allows the cover (300) to move from an extended position to a retracted position. As shown with reference to FIG. 3, the compressible portion, of the cover (300) is shown as hinge portions or elements (316a, b and 317a, b), which are configured for being in an extended position (See FIG. 3a), in a retracted position where the hinge portions flex outward away from the tissue engaging member (330) (See FIG. 3B), and in a collapsed position where the hinge portion (316a, b and 317a, b) are collapsed inward toward the tissue engaging member (330) (See FIG. 3C).

Accordingly, with reference to the embodiment of FIG. 3B, when the cover (300) is in a retracted position, the hinge portions (316a, b and 317a, b) are flexed outward away from the tissue engaging member (330) and the tissue engaging member (330) is exposed and extends beyond a distal portion of the cover (300). With reference to FIG. 3C, when the cover (300) is in an extended position, the hinge portions are in an extended position and the cover (300) encases at least a distal portion of the tissue engaging member (330), thereby preventing it from being exposed. When in the extended position, the hinge portions (316a, b and 317a, b) may be collapsed toward one another and the tissue engaging member (330), thereby locking the cover (300) in place and preventing any further axial movement of the cover (300) along the longitudinal axis of the tissue engaging member (300).

In another embodiment, a tissue engaging device with a retractable and protective cover, as described above, is provided for the collection of a sample or the delivery of a medicament. Accordingly, in certain embodiments, the tissue engaging device is a sample collecting device, such as a lancing device or a syringe, which is configured for collecting a fluid sample, for instance, a blood sample. In this embodiment, the tissue engaging member may be a lance or a needle, for instance, a needle with a beveled tip. The needle may contain a lumen through which the fluid (e.g., blood) is collected.

In certain embodiments, the body or holding member may be configured for collecting a fluid sample (e.g., blood) that is withdrawn via the lance or needle once the protective cover (as described above) is moved into a retracted position and the tissue engaging member is exposed and contacted with a tissue. For instance, the body may be a rectangular or circular body of a lancing device and may contain a lumen that may or may not include an analyte sensor such as a test strip (e.g., a removable test strip). The body may also be a barrel, for instance, the barrel of a test-tube or syringe that is associated with the tissue engaging member (e.g., a needle) and configured for withdrawing a bodily fluid, such as blood, lymph, or the like. In certain embodiments the tissue engaging device is a syringe, as described above, however the syringe is configured for delivering a flowable solution (e.g., a medicament) through the tissue engaging member (e.g., a beveled needle) to a subject once the cover is moved into a retracted position around the needle.

In certain embodiments, a device of the invention is an integrated device, such as an integrated lancing device. For instance, the integrated device may include one or more of an analyte sensor or a plurality of sensors, a meter, a motor, and the like. In one embodiment, at least one analyte sensor is provided, wherein the analyte sensor(s) is capable of indicating the level or amount of an analyte present in a bodily fluid (e.g., the blood) once the bodily fluid is collected from the tissue engaging member. The analyte sensor, for example, may be contained within a cartridge of the device (e.g., within a cartridge of the base member). The device may further include a user interface that allows a user to read the sensor.

In one embodiment, both tissue engaging member and the base member include a lumen. The lumen of the base member further includes a sensor. Therefore, during use, the tissue engaging member collects the bodily fluid (e.g., blood) and passes it through the lumen of the tissue engaging member and into the lumen of the base member. Once in the lumen of the base member the collected sample is contacted with the analyte sensor. Any analyte sensor for detecting any analyte in a fluid or blood sample may be used as is well known in the art. For instance, an analyte sensor may be a test strip that is capable of indicating the amount or level of glucose in a blood sample. Accordingly, the collection and testing of the fluid sample can be performed in a single use operation. Additionally, the fluid sample (e.g., blood) may be collected and then later contacted with a sensor or other analyte test element. For instance, the device may include a tissue engaging member that does not include a lumen, and therefore, once used to collect a sample the tissue engaging member is then contacted with an analyte sensor (e.g., a test strip).

Additionally, an integrated device of the invention may include a moveable tissue engaging member and a motor or drive mechanism operatively connected to a proximal portion of the tissue engaging member and configured for moving the tissue engaging member from a retracted to an extended position so as to pierce through the protective cover. Accordingly, when in a retracted position the moveable tissue engaging member is bounded by and encased within the protective cover. However, upon activation of the drive mechanism, the tissue engaging member is automatically moved to an extended position, whereby the distal portion of the tissue engaging member pierces through the cover (e.g., the soft material body of the cover) and is thereby exposed.

Also provided are methods of using a tissue engaging device (for instance, a lancing device or syringe) of the invention. In one embodiment, the method involves exposing a tissue engaging member. For instance, the method may involve moving a retractable and protective cover of the invention from an extended position (e.g., a first extended position) into a retracted position to expose a tissue engaging member of the device. The method may include contacting the exposed tissue engaging member with a tissue and collecting a sample (e.g., a blood sample) or delivering a fluid (e.g., a medicament) by manipulating the device in an appropriate manner so as to effectuate the task for which the device was designed (e.g., collecting a sample, delivering a medicament, making an incision, or the like). Once used, the cover may be moved from the retracted position to the extended position (e.g., the same extended position as before or a second extended position), thereby covering the tissue engaging member, and locked in place about the tissue engaging member in a manner sufficient to prevent accidental contact with the tissue engaging portion of the tissue engaging member. Once locked (e.g., before or after use) further axial movement of the cover along the longitudinal axis of the tissue engaging member is prevented.

Accordingly, the devices of the invention may be used to collect a fluid sample from a subject. In one embodiment, the method involves exposing a tissue engaging member. For instance, a method of collecting a sample from a subject includes retracting the retractable protective cover thereby allowing the tissue engaging member to pierce through the soft material body of the cover and to become exposed, contacting a tissue with the tissue engaging member, and collecting a sample. The sample may be contacted with a sensor element and the cover is then extended so as to cover the distal portion of the tissue engaging member. Once the cover covers the distal portion of the tissue engaging member the cover is locked thereby preventing the tissue engaging member from being exposed.

Where the device includes a sensor, the contacting of the fluid sample with the sensor element may occur substantially automatically, e.g., via capillary action. Where the sensor is not associated with a sensor, the sample containing tissue engaging element may be contacted directly with an external sensor which is then contacted with the sensor. Where the device includes a meter, the methods of the invention may further include reading the meter.

Additionally, the devices of the invention may be used to deliver a fluid agent (e.g., a medicament) to a subject. A method of delivering an agent to a subject includes retracting the retractable protective cover thereby allowing the tissue engaging member to pierce through the soft material body of the cover and to become exposed, contacting a tissue with the tissue engaging member, and delivering the agent. Once delivered, the cover is then extended so as to cover the distal portion of the tissue engaging member and locked thereby preventing the tissue engaging member from being exposed.

A variation on the above embodiments involve contacting a portion of the skin with the cover of the device (i.e., before removing the cover), applying a small force to the base of the device (which is enough to cause the cover to retract with respect to the tissue engaging member) to expose the tissue engaging member (i.e., applying a force sufficient to cause the tissue engaging member to pierce through the cover), contacting the exposed the tissue engaging member to the skin, and collecting and/or delivering a sample.

A method of fabricating a tissue engaging device of the invention is also provided. In one embodiment, the method involves fabricating a device that includes a cover, a tissue engaging member, and a base member. Embodiments include forming a cover of a soft material or a soft material and relatively rigid material. Certain embodiments include coextruding soft polymer material (e.g., TPE) with more rigid material to provide a cover for a tissue engaging member. Embodiments also include laminating soft material (e.g., TPE) between thin layers of rigid plastic and/or metal. Other manners of forming a cover may also be employed. Once a cover is fabricated, various features may be formed, e.g., to enable the cover to move about the tissue engaging member to cover and expose the member and/or lock the cover to the base member, as described herein.

Once fabricated the cover or more specifically the soft material body is contacted with the structural element in such a manner that the structural element bounds the soft material body. The tissue engaging member is then embedded within the soft polymer and associated with the base member (or vice versa). The resultant device is then sterilized, for instance, once the tissue engaging member is embedded within in the soft material body. Any means of sterilization is suitable so long as it does not cause substantial breakdown of the structural element or soft material body. For instance, the device may be sterilized via irradiation, such as gamma or electron beam radiation, heat (such as autoclaving) or the like. It is understood that although the above description was set forth in a given sequence of steps these steps may be interchanged without departing from the nature of the invention.

Also provided are kits for use in practicing methods of the invention. Generally, representative kits of the invention may include one or more covers configured for being movably attached to a lancing device, syringe or other medical or dental instrument and/or locked into place about a tissue engaging member of the medical or dental device. Specifically, a kit of the invention may include one or more lancing devices or syringes with retractable and protective covers that are configured for collecting or delivering a sample. For instance, a kit of the invention may include a lancing device or a syringe with a retractable and protective cover, as described above, wherein the cover is configured for being retracted, extended and/or locked. The device may include an internal sensor, meter or motor or the kit may include one or more of an external sensor element, one or more test strips, a motor, and/or a meter that is to be associated with the device. The kit may include one or more of the following components: a structural element, soft material body, a tissue engaging member (e.g., a lance, needle, blade or the like), an analyte test strip, cartridge, sensor, meter or the like. The kit may further include instructions as to how to assemble and/or use a device of the invention.

While a number of exemplary embodiments have been particularly described, those skilled in the art of medical and dental device fabrication and use will appreciate that an unlimited number of device configurations are adaptable for use with the methods provided herein and are, therefore, within the scope of the present invention. The suitability of a particular device configuration, for instance, for collecting a sample, delivering a medicament, making an incision, and the numerous permutations thereof, will depend on the particularities of the indication(s) being treated and the particular use to which the practitioner wishes to make of the device. In other words, any suitable medical or dental device, for instance, lancing device, syringe, scalpel, or the like may be configured for use with the retractable and protective covers of the invention with the appropriate modifications, which are well within the skill set of the skilled practitioner.

It is evident from the above description that embodiments of the subject invention provide a fail-proof cover with a number of advantages including, but not limited to one or more of, minimizing the risk of accidental contact causing injury, the spread of disease, loss of the protective cover, as well as preventing the reuse of a previously used device wherein secondary use is deemed to be non-beneficial. As such, the subject invention represents a significant contribution to the field of medical and dental devices.

It is to be understood that this invention is not limited to particular embodiments described above, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

While the present invention has been described with reference to the specific embodiments thereof, it should be understood by those skilled in the art that various changes may be made and equivalents may be substituted without departing from the true spirit and scope of the invention. In addition, many modifications may be made to adapt to a particular indication, material, and composition of matter, process, process step or steps, while achieving the objectives, spirit and scope of the present invention. All such modifications are intended to be within the scope of the claims appended hereto.

What is claimed is:

1. A tissue engaging device, comprising:
a tissue engaging member;
a base member from which the tissue engaging member extends; and
a compressible cover having an attachment end attached to the base member and a distal end opposite the attachment end, wherein the distal end is axially movable along a longitudinal axis of the tissue engaging member to expose the tissue engaging member while the attachment end is stationary.

2. The tissue engaging device of claim 1, wherein the compressible cover has an extended position encasing at least a distal portion of the tissue engaging member, a retracted position exposing at least the distal portion of the tissue engaging member, and a locked position preventing the distal end of the compressible cover from axial movement along the longitudinal axis.

3. The tissue engaging device of claim 2, wherein the compressible cover comprises a hinge element located at a position between the attachment end and the distal end.

4. The tissue engaging device of claim 3, wherein the hinge element bows inward toward the longitudinal axis of the tissue engaging member in the locked position.

5. The tissue engaging device of claim 3, wherein when the hinge element is in the retracted position, the hinge element is flexed outward away from the tissue engaging member.

6. The tissue engaging device of claim 1, wherein the tissue engaging member comprises a blade, a lance or a needle.

7. The tissue engaging device of claim 1, wherein the cover comprises a soft polymer portion which is piercable by the tissue engaging member.

8. The tissue engaging device of claim 7, wherein the soft polymer portion comprises an elastomer.

9. The tissue engaging device of claim 8, wherein the elastomer comprises a thermoplastic elastomer.

10. The tissue engaging device of claim 7, wherein the soft polymer portion bounds at least a distal portion of the tissue engaging member.

11. The tissue engaging device of claim 1, wherein the base member has a planar configuration with distal, proximal and side edges being linear, the side edges at substantially right angles to the distal and proximal edges.

12. The tissue engaging device of claim 1, wherein the cover comprises a first structure extending along the longitudinal axis from one side of the base member and a second structure extending along the longitudinal axis from an opposite side of the base member.

13. The tissue engaging device of claim 12, wherein the each of the first structure and second structure comprise a hinge element configured for being extended, retracted and collapsed.

14. The tissue engaging device of claim 12, wherein when the hinge elements are collapsed in a collapsed position, the cover covers at least a distal portion of the tissue engaging member and the distal end is prevented from moving along the longitudinal axis of the tissue engaging member.

15. The tissue engaging device of claim 14, wherein the hinge elements bow inward toward the longitudinal axis of the tissue engaging member in the collapsed position.

16. The tissue engaging device of claim 13, wherein when the hinge elements are retracted in the retracted position, the hinge elements are flexed outward in opposing directions from the tissue engaging member.

17. A method of lancing tissue with a tissue engaging device comprising a base member, a tissue engaging member extending from the base member and a compressible cover having an attachment end attached to the base member and a distal end opposite the attachment end, the method comprising:
moving the distal end of the compressible cover from an extended position in which the distal end encases at least a portion of a tissue engaging member, to a retracted position in which the distal end exposes the tissue engaging member while the attachment end is stationary;
contacting tissue with the exposed tissue engaging member; and
moving the distal end from the retracted position back to the extended position to encase at least a distal portion of the tissue engaging member.

18. The method of claim 17, further comprising:
locking the cover in a locked position to prevent axial movement of the distal end.

19. The method of claim 18, wherein the compressible cover comprises a hinge element located at a position between the attachment end and the distal end, and wherein locking the cover comprises bowing the hinge element inward toward a longitudinal axis of the tissue engaging member.

20. The method of claim 19, wherein moving the distal end to the retracted position comprises flexing the hinge element outward away from the tissue engaging member.
